(19) United States
(12) Patent Application Publication
(10) Pub. No.: US 2024/0180823 A1
(43) Pub. Date: Jun. 6, 2024

(54) INHALATION SPRAY OF SEMAGLUTIDE AND PREPARATION METHOD THEREOF

(71) Applicant: QILU PHARMACEUTICAL CO., LTD., Shandong (CN)

(72) Inventors: Min ZHANG, Shandong (CN); Qingmin YANG, Shandong (CN); Yongli TAN, Shandong (CN); Ping HE, Shandong (CN); Shuaishuai WANG, Shandong (CN); Guiming LIN, Shandong (CN); Xiaoqing CHENG, Shandong (CN)

(73) Assignee: QILU PHARMACEUTICAL CO., LTD., Shandong (CN)

(21) Appl. No.: 18/527,129

(22) Filed: Dec. 1, 2023

(30) Foreign Application Priority Data
Dec. 5, 2022 (CN) 202211547056.X

(51) Int. Cl. A61K 9/00; A61K 38/26; A61K 47/02; A61K 47/10; A61K 47/12; A61K 47/18; A61K 47/26; A61M 11/00

(52) U.S. Cl. CPC A61K 9/0073; A61K 38/26; A61K 47/02; A61K 47/10; A61K 47/12; A61K 47/186; A61K 47/26; A61M 11/001

(57) ABSTRACT

The present invention provides an inhalation spray of Semaglutide with good patient compliance and high bioavailability at the targeted site, which belongs to the technical field of pharmaceutical preparation. The inhalation spray of Semaglutide according to the invention comprising, by weight to volume, 0.01% to 20% of Semaglutide in the inhalation spray, wherein the proportion of fine particles with a particle size less than 5 μm formed after the inhalation spray is atomized by a spray device is not less than 10%. The inhalation spray of Semaglutide according to the invention has a simple formulation, low toxic and side effects, atomization administration of the medicinal liquid through an inhalation device, uniform distribution of spray droplets, a high lung deposition rate, low irritation and convenient administration, which is suitable for industrial production. The invention further provides a preparation method of the inhalation spray of Semaglutide.

This patent application claims the benefit of priority of a Chinese application No. 202211547056.X, filed on Dec. 5, 2022, which is incorporated herein by reference in its entirety.

### TECHNICAL FIELD

The present invention belongs to the technical field of pharmaceutical preparation, and relates to an inhalation spray of Semaglutide and preparation method thereof.

### BACKGROUND

An inhalation spray refers to a solution, suspension or emulsion that produces an aerosol for inhalation by a predetermined or quantitative atomizer. When in use, the contents need to be released as a mist with a help of pressure of a manual pump, high-pressure gas, ultrasonic vibration or other methods, so that a certain amount of atomized liquid can be inhaled in the form of aerosol during a single respiration. Due to an accurate dose, stable dose, small droplets released, slow running speed and long duration of the inhalation spray, the inhalable time of medicament and the deposition rate in lung are improved, and the compliance of patient is good.

Semaglutide is an agonist of glucagon-like peptide-1 (GLP-1) receptor, which can bind to and activate GLP-1 receptor, stimulate insulin secretion in a glucose-dependent manner by mediating GLP-1 receptor, and reduce glucagon secretion, thereby achieving an effect of reducing blood glucose. The long-acting mechanism of Semaglutide lies in that the lysine at position 26 of the peptide chain is connected to the C18 fatty diacid side chain, which enhances the affinity for albumin, avoids rapid clearance by kidney, prevents metabolic degradation, and thus prolongs the half-life in vivo; the position 8 of the peptide chain is modified to α-aminoisobutyric acid to avoid degradation by DPP-4 enzyme, which increases the stability; and the lysine at position 34 is replaced by arginine to ensure the stability of the C18 side chain at position 26.

At present, Semaglutide injection (trade name of Ozempic®) and oral Semaglutide in a tablet (trade name of Rybelsus®) developed by Novo Nordisk are on the market at home and abroad. Semaglutide injections with specifications of 2 mg/1.5 mL and 4 mg/3 mL were first marketed in the United States in December 2017, were approved for marketing in China in April 2021, and have been marketed in more than 50 countries or regions such as Europe, Canada and Japan so far. Semaglutide injection is injected subcutaneously with dosages of 0.25 mg, 0.5 mg and 1 mg, once a week, which is used to control blood glucose in type II diabetes. Due to an inconvenient administration of the injection, patients have a relatively great psychological obstacle, and thus oral Semaglutide in a tablet administrated once a day was marketed by the original research company in the United States in September 2019. However, due to low oral bioavailability, the dosage was increased to 3 mg, 7 mg and 14 mg, while the absolute bioavailability was only 0.4% to 1%, and the absorption effect was poor.

There are a large amount of propylene glycol isoosmotic agent (1.4%) in the formulation of Semaglutide injection, which may lead to certain toxic and side effects. Additionally, the injection administration is inconvenient, and the patient's compliance thereof is poor. Oral Semaglutide in tablet has complex formulation, low bioavailability, large dosage and inconvenient administration, of which drug absorption is seriously affected by diet, so that there are explicit requirements for time and mode of taking medicament. The medicament is required to be taken at least 30 minutes before the first diet and with no more than 4 ounces of boiled water every day. A waiting time of less than or more than 30 minutes will affect the absorption of tablets. Therefore, there is an urgent need for a new preparation of Semaglutide with convenient administration, high bioavailability and good patient compliance to replace the injection and the oral tablet.

Due to the large specific surface area of human pulmonary alveoli, rich capillary network, thin alveolar epithelial cell layer and low biological enzyme activity, inhalation spray can be quickly absorbed and maintain biological activity after oral inhalation, with high bioavailability, convenient administration and low irritation and without a first-pass effect. The inhalation spray is expected as a preferred administration mode of Semaglutide to replace the injection and the oral tablet.

The viscosity of a medicinal solution of Semaglutide increases with the increase of concentration, and the spray droplet size distribution of the medicinal liquid with high concentration is wide. Therefore, it is difficult to ensure effective deposition of medicinal droplets in lung. Polypeptide drugs have large molecular weight, and there may be some obstacles in transmembrane absorption. Therefore, in order to achieve efficient deposition and effective absorption of Semaglutide in lung, control of Semaglutide concentration in the medicinal liquid and the spray droplet size distribution, and how to improve the transmembrane bioavailability of the drug are the problems that need to be solved.

### SUMMARY OF THE INVENTION

The invention aims to provide a new preparation of Semaglutide with good patient compliance and high bioavailability at the targeted site: inhalation spray.

The inhalation spray of Semaglutide according to the invention has a simple formulation, low toxic and side effects, atomization administration of the medicinal liquid through an inhalation device, uniform distribution of spray droplets, a high lung deposition rate, low irritation and convenient administration, which is suitable for industrial production.

For inhalation spray of Semaglutide according to the invention, by weight to volume (g/100 ml), Semaglutide accounts for 0.01% to 20% of the inhalation spray, wherein the proportion of fine particles with a particle size less than 5 μm formed after the inhalation spray is atomized by a spray device is not less than 10%;
preferably, the proportion of fine particles with the particle size less than 5 μm formed after the inhalation spray is atomized by the spray device is not less than 20%; and
more preferably, the proportion of fine particles with the particle size less than 5 μm formed after the inhalation spray is atomized by the spray device is not less than 30%.

For the inhalation spray according to the invention, a higher proportion of fine particle content ensures the pulmonary deposition and absorption of the drug. A good therapeutic effect can be achieved by the inhalation spray after oral inhalation, which provides convenient administration, high bioavailability and good patient compliance under the premise of ensuring the therapeutic effect.

For the inhalation spray according to the invention, a mass median aerodynamic diameter (MMAD) of the fine particles formed after the inhalation spray is atomized by a spray device is not more than 10 μm. After inhalation, the spray droplets with relatively small particle size can effectively enter the pulmonary alveolus and be absorbed into the blood, exerting a therapeutic effect.

In some preferred embodiments, Semaglutide accounts for 0.01% to 15% of the inhalation spray of Semaglutide according to the invention, by weight to volume.

The inhalation spray according to the invention further comprises 0.01% to 2% of a pharmaceutically acceptable bacteriostatic agent by weight to volume, wherein the bacteriostatic agent is selected from the group consisting of phenol, benzyl alcohol, benzalkonium chloride, m-phenol, sodium benzoate, chlorobutanol, butyl hydroxybenzoate, methyl hydroxybenzoate and a combination thereof; preferably, the bacteriostatic agent is selected from the group consisting of phenol, benzyl alcohol, benzalkonium chloride and a combination thereof.

The inhalation spray according to the invention further comprises a pharmaceutically acceptable bacteriostatic agent. In some preferred embodiments, the bacteriostatic agent is phenol or benzyl alcohol, which accounts for, by weight to volume:
0.45% to 0.65% of the inhalation spray, when the bacteriostatic agent is phenol; or
0.5% to 1.5% of the inhalation spray, when the bacteriostatic agent is benzyl alcohol.

In some embodiments, the inhalation spray according to the invention further comprises 0% to 15% of an absorption enhancer, by weight to volume. In some preferred embodiments, the absorption enhancer accounts for 0.01% to 10%.

For the inhalation spray according to the invention, in some preferred embodiments, the absorption enhancer is selected from the group consisting of Tween 20, Tween 80, sodium octanoate, sodium deoxycholate, sodium dodecyl sulfate, sucrose laurate, sodium 8-(2-hydroxybenzamido)octanoate and a combination thereof. In some more preferred embodiments, the absorption enhancer is selected from the group consisting of Tween 80, sodium octanoate, sucrose laurate and a combination thereof.

The inhalation spray according to the invention further comprises an absorption enhancer. In some still more preferred embodiments, the absorption enhancer is Tween 80 with a content of 0.01% to 0.15%, by weight to volume.

In some embodiments, the inhalation spray according to the invention further comprises a pH buffer selected from the group consisting of disodium phosphate, sodium biphosphate, sodium citrate, sodium acetate, sodium carbonate, sodium tartrate and a combination thereof. In some preferred embodiments, the pH buffer is selected from the group consisting of disodium phosphate, sodium biphosphate and a mixture thereof.

In some preferred embodiments, the inhalation spray according to the invention has a pH of 7.1 to 7.7. In some more preferred embodiments, the inhalation spray has a pH of 7.3 to 7.5. Preferably, the pH value of the spray can be adjusted with hydrochloric acid or sodium hydroxide. Other suitable pH regulators such as sulfuric acid, sodium carbonate, sodium bicarbonate and the like can also be selected.

The invention further provides a preparation method of any of the above inhalation sprays, comprising the following steps:
(1) taking 80% of a formulation volume of water, adding a bacteriostatic agent, an absorption enhancer and a pH buffer, stirring to dissolve, and adjusting pH to 7.1 to 7.7 with sodium hydroxide and hydrochloric acid;
(2) adding Semaglutide and stirring until it is completely dissolved;
(3) adjusting pH to 7.3 to 7.5 with sodium hydroxide and hydrochloric acid; and
(4) adding water to the formulation volume.

The water in step (4) can be purified water or water for injection, and the water for injection is used in large-scale production. The prepared medicinal liquid needs to be sterilized and filtered to ensure the sterility of the medicinal liquid.

The preparation method herein has the advantages including simple process, low cost, and suitable for industrial production. The product obtained by this method has stable quality and is easy to be preserved and transported.

Compared with the existing injection and the oral tablet, the inhalation spray of Semaglutide of the invention has the following beneficial effects.
1. The invention provides a new preparation—inhalation spray of Semaglutide. Compared with the injection in the prior art, the inhalation spray of the invention has the advantages of convenient administration, reducing the pain and psychological pressure of the patients, greatly improving the compliance, and being beneficial to clinical use. Compared with the tablets in the prior art, the inhalation spray of the invention has the advantages including simple formulation, avoiding the serious influence of food on drug absorption, convenient administration, high bioavailability and good safety.
2. The pharmaceutical composition of the invention has small spray particle size and high lung deposition rate. Especially, when Tween 80 and benzyl alcohol are used as adjuvants, not only drug absorption and bacteriostasis are promoted, but also the spray particle size is reduced, and the minimum spray particle size MMAD is about 5 μm, which is particularly suitable for inhalation administration. The proportion of fine particles of spray droplets with particle size less than 5 μm formed after the liquid medicament is atomized by the inhalation device is not less than 10%, which can be up to 20%, 30% or even more than 40% in preferred embodiments. A higher proportion of fine particles ensures the drug deposition in the lung.
3. In the pharmaceutical composition of the invention, the use of absorption enhancers such as Tween 80, sodium octanoate, or sucrose laurate significantly improves the transmembrane absorption efficiency of the drug, so that the bioavailability of the pharmaceutical composition of the invention reaches 37.1% to 52.1% during inhalation administration.
4. The pharmaceutical composition of the invention does not comprise osmotic pressure regulators such as propylene glycol, thereby avoiding side effects caused by the use of a large number of osmotic pressure regulators.

### BRIEF DESCRIPTION OF THE DRAWINGS

FIG. 1 shows bioavailabilities of airway administration with different formulations of inhalation sprays (Examples 3 to 7 and 12) relative to bioavailability of subcutaneous injection in rats.

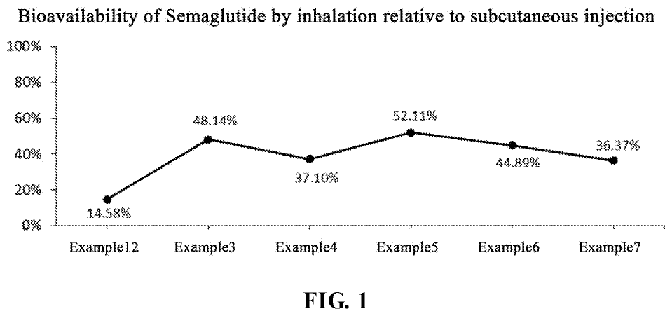

### DETAILED DESCRIPTION

The present invention is further illustrated by a plurality of the following specific Examples. It should be understood that the examples of the present invention are merely illustrative of the present invention and are not to limit the present invention. On the basis of the technical solution of the present invention, the technical solutions obtained by simply modifying the present invention or equivalent replacement of the present invention using conventional means or active components all belong to the protection scope of the present invention.

### Example 1

The inhalation spray of Semaglutide at a concentration of 2% with phenol as bacteriostatic agent was prepared. The specific formulation composition is shown in Table 1.

TABLE 1

| Formulation composition | | Amount of 50 mL | |
|---|---|---|---|
| Active ingredient | Semaglutide | 1 | g |
| Adjuvants | anhydrous disodium phosphate | 56.5 | mg |
| | phenol | 275 | mg |

| | |
|---|---|
| sodium hydroxide | q.s. |
| hydrochloric acid | q.s. |
| purified water | to 50 mL |

### Preparation Process:

40 mL of purified water was taken, to which the formulation amount of anhydrous disodium phosphate and phenol were added respectively. The system was stirred until dissolution, the pH of which was detected to be 9.1 and then adjusted to 7.5 with dilute hydrochloric acid. 1 g of Semaglutide was added, and the system was stirred until dissolution, the pH of which was adjusted to 7.4 with sodium hydroxide. Purified water was added to a constant volume of 50 mL, then the pH was detected to be 7.4, and an inhalation spray of Semaglutide at a concentration of 2% was obtained.

### Example 2

The inhalation spray of Semaglutide at a concentration of 10% with benzalkonium chloride as bacteriostatic agent was prepared. The specific formulation composition is shown in Table 2.

TABLE 2

| | | Amount of | |
|---|---|---|---|
| Formulation composition | | 50 mL dosage | |
| Active ingredient | Semaglutide | 5 | g |
| Adjuvants | anhydrous disodium | 56.5 | mg |
| | phosphate | | |
| | benzalkonium chloride | 5 | mg |

| | |
|---|---|
| sodium hydroxide | q.s. |
| hydrochloric acid | q.s. |
| purified water | to 50 mL |

### Preparation Process:

40 mL of purified water was taken, to which the formulation amount of anhydrous disodium phosphate and benzalkonium chloride were added respectively. The system was stirred until dissolution, the pH of which was detected to be 9.0 and then adjusted to 7.5 with dilute hydrochloric acid. 5 g of Semaglutide was added, and the system was stirred until dissolution, the pH of which was adjusted to 7.4 with sodium hydroxide. Purified water was added to a constant volume of 50 mL, then the pH was detected to be 7.5, and an inhalation spray of Semaglutide at a concentration of 10% was obtained.

### Example 3

The inhalation spray of Semaglutide at a concentration of 7.5% with phenol as bacteriostatic agent and sodium octanoate as absorption enhancer was prepared. The specific formulation composition is shown in Table 3.

TABLE 3

| Preparation composition | | Amount of 50 mL | |
|---|---|---|---|
| Active ingredient | Semaglutide | 3.75 | g |
| Adjuvants | anhydrous disodium | 56.5 | mg |
| | phosphate | | |
| | phenol | 275 | mg |
| | sodium octanoate | 5 | g |

| | |
|---|---|
| sodium hydroxide | q.s. |
| hydrochloric acid | q.s. |
| purified water | to 50 mL |

### Preparation Process:

40 mL of purified water was taken, to which the formulation amount of anhydrous disodium phosphate, phenol and sodium octanoate were added respectively. The system was stirred until dissolution, the pH of which was detected to be 8.9 and then adjusted to 7.5 with dilute hydrochloric acid. 3.75 g of Semaglutide was added, and the system was stirred until dissolution, the pH of which was detected to be 7.4. Purified water was added to a constant volume of 50 mL, then the pH was detected to be 7.3, and an inhalation spray of Semaglutide at a concentration of 7.5% was obtained.

### Example 4

The inhalation spray of Semaglutide at a concentration of 7.5% with phenol as bacteriostatic agent and sucrose laurate as absorption enhancer was prepared. The specific formulation composition is shown in Table 4.

TABLE 4

| Preparation composition | | Amount of 50 mL | |
|---|---|---|---|
| Active ingredient | Semaglutide | 3.75 | g |
| Adjuvants | anhydrous disodium | 56.5 | mg |
| | phosphate | | |
| | phenol | 275 | mg |
| | sucrose laurate | 0.5 | g |

| | |
|---|---|
| sodium hydroxide | q.s. |
| hydrochloric acid | q.s. |
| purified water | to 50 mL |

### Preparation Process:

40 mL of purified water was taken, to which the formulation amount of anhydrous disodium phosphate, phenol and sucrose laurate were added respectively. The system was stirred until dissolution, the pH of which was detected to be 7.3. 3.75 g of Semaglutide was added, and the system was stirred until dissolution, the pH of which was detected to be 7.1. Purified water was added to a constant volume of 50 mL, then the pH was detected to be 7.1, and an inhalation spray of Semaglutide at a concentration of 7.5% was obtained.

### Example 5

The inhalation spray of Semaglutide at a concentration of 7.5% with benzyl alcohol as bacteriostatic agent and Tween 80 as absorption enhancer was prepared. The specific formulation composition is shown in Table 5.

TABLE 5

| Preparation composition | | Amount of 50 mL | |
|---|---|---|---|
| Active ingredient | Semaglutide | 3.75 | g |
| Adjuvants | anhydrous disodium | 56.5 | mg |
| | phosphate | | |
| | benzyl alcohol | 500 | mg |
| | Tween 80 | 10 | mg |

| | |
|---|---|
| sodium hydroxide | q.s. |
| hydrochloric acid | q.s |
| purified water | to 50 mL |

### Preparation Process:

40 mL of purified water was taken, to which the formulation amount of anhydrous disodium phosphate, benzyl alcohol and Tween 80 were added respectively. The system was stirred until dissolution, the pH of which was detected to be 8.9 and then adjusted to 7.5 with dilute hydrochloric acid. 3.75 g of Semaglutide was added, and the system was stirred until dissolution, the pH of which was adjusted to 7.4 with sodium hydroxide. Purified water was added to a constant volume of 50 mL, then the pH was detected to be 7.4, and an inhalation spray of Semaglutide with a concentration of 7.5% was obtained.

### Example 6

The inhalation spray of Semaglutide at a concentration of 7.5% with benzyl alcohol as bacteriostatic agent and Tween 80 and sodium octanoate as absorption enhancer was prepared. The specific formulation composition is shown in Table 6.

TABLE 6

| Preparation composition | | Amount of 50 mL | |
|---|---|---|---|
| Active ingredient | Semaglutide | 3.75 | g |
| Adjuvants | anhydrous disodium | 56.5 | mg |
| | phosphate | | |
| | benzyl alcohol | 500 | mg |
| | Tween 80 | 10 | mg |
| | sodium octanoate | 5 | g |

| | |
|---|---|
| sodium hydroxide | q.s. |
| hydrochloric acid | q.s. |
| purified water | to 50 mL |

### Preparation Process:

40 mL of purified water was taken, to which the formulation amount of anhydrous disodium phosphate, benzyl alcohol, Tween 80 and sodium octanoate were added respectively. The system was stirred until dissolution, the pH of which was detected to be 8.9 and then adjusted to 7.5 with dilute hydrochloric acid. 3.75 g of Semaglutide was added, and the system was stirred until dissolution. Purified water was added to a constant volume of 50 mL, then the pH was detected to be 7.4, and an inhalation spray of Semaglutide with a concentration of 7.5% was obtained.

### Example 7

The inhalation spray of Semaglutide at a concentration of 7.5% with benzyl alcohol as bacteriostatic agent and Tween 80 and sucrose laurate as absorption enhancer was prepared. The specific formulation composition is shown in Table 7.

TABLE 7

| Preparation composition | | Amount of 50 mL | |
|---|---|---|---|
| Active ingredient | Semaglutide | 3.75 | g |
| Adjuvants | anhydrous disodium | 56.5 | mg |
| | phosphate | | |
| | benzyl alcohol | 500 | mg |
| | Tween 80 | 10 | mg |
| | sucrose laurate | 5 | g |

| | |
|---|---|
| sodium hydroxide | q.s. |
| hydrochloric acid | q.s. |
| purified water | to 50 mL |

### Preparation Process:

40 mL of purified water was taken, to which the formulation amount of anhydrous disodium phosphate, benzyl alcohol, Tween 80 and sucrose laurate were added respectively. The system was stirred until dissolution, the pH of which was detected to be 7.2. 3.75 g of Semaglutide was added, stirred and dissolved, the pH of which was detected to be 7.0. Purified water was added to a constant volume of 50 mL, then the pH was detected to be 7.1, and an inhalation spray of Semaglutide at a concentration of 7.5% was obtained.

### Example 8

The inhalation spray of Semaglutide at a concentration of 10% with benzyl alcohol as bacteriostatic agent and 0.02% Tween 80 as absorption enhancer was prepared. The specific formulation composition is shown in Table 8.

TABLE 8

| Preparation composition | | Amount of 50 mL | |
|---|---|---|---|
| Active ingredient | Semaglutide | 5 | g |
| Adjuvants | anhydrous disodium | 56.5 | mg |
| | phosphate | | |
| | benzyl alcohol | 500 | mg |
| | Tween 80 | 10 | mg |

| | |
|---|---|
| sodium hydroxide | q.s. |
| hydrochloric acid | q.s. |
| purified water | to 50 mL |

### Preparation Process:

40 mL of purified water was taken, to which the formulation amount of anhydrous disodium phosphate, benzyl alcohol and Tween 80 were added respectively. The system was stirred until dissolution, the pH of which was detected to be 8.9 and then adjusted to 7.5 with dilute hydrochloric acid. 5 g of Semaglutide was added, and the system was stirred until dissolution, the pH of which was adjusted to 7.3 with sodium hydroxide. Purified water was added to a constant volume of 50 mL, then the pH was detected to be 7.3, and an inhalation spray of Semaglutide at a concentration of 10% was obtained.

### Example 9

The inhalation spray of Semaglutide at a concentration of 10% with benzyl alcohol as bacteriostatic agent and 0.1% Tween 80 as absorption enhancer was prepared. The specific formulation composition is shown in Table 9.

TABLE 9

| Preparation composition | | Amount 50 mL | |
|---|---|---|---|
| Active ingredient | Semaglutide | 5 | g |
| Adjuvants | anhydrous disodium | 56.5 | mg |
| | phosphate | | |
| | benzyl alcohol | 500 | mg |
| | Tween 80 | 50 | mg |

| | |
|---|---|
| sodium hydroxide | q.s. |
| hydrochloric acid | q.s. |
| purified water | to 50 mL |

### Preparation Process:

40 mL of purified water was taken, to which the formulation amount of anhydrous disodium phosphate, benzyl alcohol and Tween 80 were added respectively. The system was stirred until dissolution, the pH of which was detected to be 8.9 and then adjusted to 7.5 with dilute hydrochloric acid. 5 g of Semaglutide was added, and the system was stirred until dissolution, the pH of which was adjusted to 7.4 with sodium hydroxide. Purified water was added to a constant volume of 50 mL, then the pH was detected to be 7.3, and an inhalation spray of Semaglutide at a concentration of 10% was obtained.

### Example 10

The inhalation spray of Semaglutide at a concentration of 10% with benzyl alcohol as bacteriostatic agent and 0.15% Tween 80 as absorption enhancer was prepared. The specific formulation composition is shown in Table 10.

TABLE 10

| Preparation composition | | Amount of 50 mL | |
|---|---|---|---|
| Active ingredient | Semaglutide | 5 | g |
| Adjuvants | anhydrous disodium | 56.5 | mg |
| | phosphate | | |
| | benzyl alcohol | 500 | mg |
| | Tween 80 | 75 | mg |

| | |
|---|---|
| sodium hydroxide | q.s. |
| hydrochloric acid | q.s. |
| purified water | to 50 mL |

### Preparation Process:

40 mL of purified water was taken, to which the formulation amount of anhydrous disodium phosphate, benzyl alcohol and Tween 80 were added respectively. The system was stirred until dissolution, the pH of which was detected to be 9.0 and then adjusted to 7.6 with dilute hydrochloric acid. 5 g of Semaglutide was added, and the system was stirred until dissolution, the pH of which was adjusted to 7.6 with sodium hydroxide. Purified water was added to a constant volume of 50 mL, then the pH was detected to be 7.7, and an inhalation spray of Semaglutide at a concentration of 10% was obtained.

### Example 11

The inhalation spray of Semaglutide at a concentration of 15% with benzyl alcohol as bacteriostatic agent and 0.02% Tween 80 as absorption enhancer was prepared. The specific formulation composition is shown in Table 11.

TABLE 11

| Preparation composition | | Amount of 50 mL | |
|---|---|---|---|
| Active ingredient | Semaglutide | 7.5 | g |
| Adjuvants | anhydrous disodium | 56.5 | mg |
| | phosphate | | |
| | benzyl alcohol | 500 | mg |
| | Tween 80 | 10 | mg |

| | |
|---|---|
| sodium hydroxide | q.s. |
| hydrochloric acid | q.s. |
| purified water | to 50 mL |

### Preparation Process:

40 mL of purified water was taken, to which the formulation amount of anhydrous disodium phosphate, benzyl alcohol and Tween 80 were added respectively. The system was stirred until dissolution, the pH of which was detected to be 8.9 and then adjusted to 7.5 with dilute hydrochloric acid. 7.5 g of Semaglutide was added, and the system was stirred until dissolution, the pH of which was adjusted to 7.3 with sodium hydroxide. Purified water was added to a constant volume of 50 mL, then the pH was detected to be 7.3, and an inhalation spray of Semaglutide at a concentration of 15% was obtained.

### Comparative Example 1

The inhalation spray of Semaglutide at a concentration of 20% was prepared. The specific formulation composition is shown in Table 12.

TABLE 12

| Preparation composition | | Amount of 50 mL | |
|---|---|---|---|
| Active ingredient | Semaglutide | 10 | g |
| Adjuvants | anhydrous disodium | 56.5 | mg |
| | phosphate | | |
| | phenol | 275 | mg |

| | |
|---|---|
| sodium hydroxide | q.s. |
| hydrochloric acid | q.s. |
| purified water | to 50 mL |

### Preparation Process:

40 mL of purified water was taken, to which the formulation amount of anhydrous disodium phosphate and phenol were added respectively. The system was stirred until dissolution, the pH of which was detected to be 9.0 and then adjusted to 7.5 with dilute hydrochloric acid. 10 g of Semaglutide was added, and the system was stirred until dissolution, the pH of which was adjusted to 7.4 with sodium hydroxide. Purified water was added to a constant volume of 50 mL, then the pH was detected to be 7.4, and an inhalation spray of Semaglutide at a concentration of 20% was obtained.

### Comparative Example 2

The inhalation spray of Semaglutide at a concentration of 10% with benzyl alcohol as bacteriostatic agent was prepared. The specific formulation composition is shown in Table 13.

TABLE 13
compositions

| Preparation compositions | | Amount of 50 mL | |
|---|---|---|---|
| Active ingredient | Semaglutide | 5 | g |
| Adjuvants | anhydrous disodium | 56.5 | mg |
| | phosphate | | |
| | benzyl alcohol | 500 | mg |

| | |
|---|---|
| sodium hydroxide | q.s. |
| hydrochloric acid | q.s |
| purified water | to 50 mL |

### Preparation Process:

40 mL of purified water was taken, to which the formulation amount of anhydrous disodium phosphate and benzyl alcohol were added respectively. The system was stirred until dissolution, the pH of which was detected to be 8.9 and then adjusted to 7.5 with dilute hydrochloric acid. 5 g of Semaglutide was added, and the system was stirred until dissolution, the pH of which was adjusted to 7.4 with sodium hydroxide. Purified water was added to a constant volume of 50 mL, then the pH was detected to be 7.4, and an inhalation spray of Semaglutide at a concentration of 10% was obtained.

### Study on Stability

Accelerated Stability Assays of the Inhalation Sprays of Semaglutide with Different Formulations:

The inhalation sprays of Semaglutide prepared in Examples 1 to 10 were placed for 0 day, 5 days and 10 days at a temperature of 40° C.±2° C. and a relative humidity of 75%±5%. The results for sampling and analysis are shown in Table 14.

TABLE 14

| | | | Related substances | | |
|---|---|---|---|---|---|
| | | | Maximum | Total | |
| | Sampling | | single | impurity | Amount |
| Examples | time | | impurity (%) | (%) | (%) |
| Example 1 | 0 | day | 0.29 | 0.45 | 99.55 |
| | 5 | days | 0.33 | 0.99 | 99.01 |
| | 10 | days | 0.46 | 1.96 | 98.04 |
| Example 2 | 0 | day | 0.15 | 0.35 | 99.65 |
| | 5 | days | 0.28 | 1.31 | 98.69 |
| | 10 | days | 0.52 | 2.72 | 97.28 |
| Example 3 | 0 | day | 0.18 | 0.33 | 99.67 |
| | 5 | days | 0.39 | 1.27 | 98.73 |
| | 10 | days | 0.80 | 2.33 | 97.67 |
| Example 4 | 0 | day | 0.18 | 0.42 | 99.58 |
| | 5 | days | 0.25 | 1.75 | 98.25 |
| | 10 | days | 0.50 | 2.94 | 97.06 |
| Example 5 | 0 | day | 0.17 | 0.46 | 99.54 |
| | 5 | days | 0.28 | 1.28 | 98.72 |
| | 10 | days | 0.45 | 2.35 | 97.65 |
| Example 6 | 0 | day | 0.16 | 0.39 | 99.61 |
| | 5 | days | 0.33 | 1.79 | 98.21 |
| | 10 | days | 0.75 | 3.33 | 96.67 |
| Example 7 | 0 | day | 0.18 | 0.43 | 99.57 |
| | 5 | days | 0.32 | 1.82 | 98.18 |
| | 10 | days | 0.47 | 3.38 | 96.62 |
| Example 8 | 0 | day | 0.15 | 0.35 | 99.65 |
| | 5 | days | 0.28 | 1.31 | 98.69 |
| | 10 | days | 0.52 | 2.72 | 97.28 |
| Example 9 | 0 | day | 0.17 | 0.36 | 99.64 |
| | 5 | days | 0.24 | 1.36 | 98.64 |
| | 10 | days | 0.47 | 2.94 | 97.06 |
| Example 10 | 0 | day | 0.14 | 0.32 | 99.68 |
| | 5 | days | 0.27 | 1.38. | 98.62 |
| | 10 | days | 0.50 | 2.70 | 97.30 |

Note:
the amount (%) is calculated as 1-total impurity (%).

The results of accelerated stability assay show that for the inhalation sprays of Semaglutide prepared according to the processes in Examples 1 to 10 were placed for 5 days and 10 days at a temperature of 40° C.±2° C. and a relative humidity of 75%±5%, the maximum impurities were below 0.80%, the total impurities were below 3.38% and the drug contents were above 96.62%. With the extension of investigation time, the maximum single impurities and total impurities tend to increase. In order to ensure stability, the product can be stored at 20 to 80.

Aerodynamic Particle Size Distribution Assays of the Inhalation Sprays of Semaglutide with Different Formulations

The inhalation sprays of Semaglutide of Examples 1 to 11 and Comparative Examples 1 to 2, which were placed for 0 day, 5 days and 10 days at a temperature of 40° C.±2° C. and a relative humidity of 75%±5%, were assembled with inhalation devices, respectively. Then according to 0951 the method of determining the aerodynamic characteristics of fine particles of inhaled preparation in Chinese Pharmacopoeia 2020 edition IV, a next generation impactor (NGI) was used to simulate the structure of human respiratory tract, and the aerodynamic particle size distribution (APSD) of spray droplets was tested to approximately evaluate the deposition of medicament particles in the lung. The assay data are shown in Table 15 (unit: μg).

TABLE 15

| Examples | | | |
|---|---|---|---|
| Example 1 | Example 2 | Example 3 | Example 4 |
| Test amount | | | |
| 2 sprays | 4 sprays | 2 sprays | 2 sprays |

Time point

| | 0 day | 5 days | 10 days | 0 day | 5 days | 10 days | 0 day | 5 days | 10 days | 0 day | 5 days | 10 days |
|---|---|---|---|---|---|---|---|---|---|---|---|---|
| Adaptor | 0.6 | 2.9 | 1.4 | 67.8 | 89.3 | 64.7 | 2.0 | 2.0 | 1.4 | 2.8 | 1.5 | 4.3 |
| Artificial | 20.0 | 24.7 | 16.7 | 1971.0 | 2584.6 | 2626.8 | 452.1 | 412.6 | 447.4 | 415.6 | 526.8 | 624.5 |
| larynx | | | | | | | | | | | | |
| stage 1 | 10.7 | 3.3 | 1.6 | 819.2 | 905.5 | 814.4 | 314.7 | 356.5 | 377.1 | 284.3 | 372.1 | 272.0 |
| stage 2 | 66.0 | 34.3 | 20.2 | 992.7 | 869.3 | 772.1 | 426.4 | 457.0 | 524.7 | 513.9 | 548.3 | 398.6 |
| stage 3 | 85.8 | 65.6 | 45.5 | 480.2 | 363.0 | 327.9 | 255.1 | 245.0 | 227.6 | 281.6 | 247.7 | 204.0 |
| stage 4 | 79.2 | 67.9 | 51.9 | 409.0 | 290.3 | 299.7 | 196.7 | 178.1 | 136.9 | 203.6 | 145.9 | 154.8 |
| stage 5 | 32.8 | 31.0 | 27.1 | 185.5 | 128.3 | 139.0 | 60.8 | 57.3 | 25.6 | 54.3 | 23.7 | 37.5 |
| stage 6 | 6.8 | 5.4 | 7.7 | 39.0 | 19.8 | 39.4 | 3.9 | 6.9 | 1.3 | 1.5 | 0.0 | 0.9 |
| stage 7 | 1.2 | 1.6 | 3.5 | 7.6 | 2.1 | 5.8 | 0.4 | 0.0 | 0.0 | 0.0 | 0.0 | 0.3 |
| MOC | 4.0 | 7.4 | 15.8 | 31.5 | 5.5 | 58.2 | 0.0 | 0.0 | 1.1 | 0.0 | 0.0 | 16.2 |
| Sum | 307.1 | 244.3 | 191.2 | 5003.5 | 5257.8 | 5147.9 | 1712.1 | 1715.1 | 1742.9 | 1757.5 | 1866.0 | 1713.1 |

| | Examples | | | | | | | | | | | |
|---|---|---|---|---|---|---|---|---|---|---|---|---|
| | Example 5 | | | Example 6 | | | Example 7 | | | Example 8 | | |
| | Test amount | | | | | | | | | | | |
| | 2 sprays | | | 2 sprays | | | 2 sprays | | | 2 sprays | | |
| | Sampling point | | | | | | | | | | | |
| | 0 day | 5 days | 10 days | 0 day | 5 days | 10 days | 0 day | 5 days | 10 days | 0 day | 5 days | 10 days |
| Adaptor | 1.2 | 0.7 | 1.9 | 2.5 | 0.6 | 2.1 | 3.4 | 2.3 | 3.3 | 2.32 | 1.79 | 3.17 |
| Artificial | 122.9 | 207.2 | 410.0 | 421.2 | 669.2 | 420.1 | 481.7 | 837.0 | 641.2 | 287.99 | 290.72 | 412.76 |
| larynx | | | | | | | | | | | | |
| stage 1 | 104.4 | 86.1 | 178.7 | 313.5 | 313.0 | 277.8 | 339.5 | 363.1 | 398.6 | 168.87 | 149.59 | 137.33 |
| stage 2 | 362.5 | 298.1 | 496.2 | 440.3 | 368.7 | 447.8 | 472.5 | 359.0 | 411.4 | 566.24 | 538.09 | 501.08 |
| stage 3 | 368.3 | 342.9 | 275.3 | 275.3 | 202.5 | 256.9 | 253.7 | 147.3 | 184.2 | 546.1 | 573.49 | 497.94 |
| stage 4 | 319.5 | 321.9 | 202.2 | 209.9 | 146.0 | 196.5 | 198.8 | 111.2 | 151.8 | 468.96 | 493.52 | 479.5 |
| stage 5 | 135.9 | 147.2 | 77.7 | 70.0 | 35.0 | 57.9 | 60.0 | 24.4 | 34.0 | 135.74 | 154.56 | 203.39 |
| stage 6 | 24.9 | 26.8 | 7.6 | 9.0 | 4.3 | 6.6 | 3.1 | 0.8 | 2.2 | 11.53 | 22.57 | 31.09 |
| stage 7 | 1.9 | 1.5 | 0.5 | 0.0 | 0.0 | 0.6 | 0.0 | 0.0 | 0.3 | 1.6 | 3.91 | 2.29 |
| MOC | 13.2 | 11.1 | 4.2 | 0.0 | 0.0 | 1.3 | 2.2 | 7.3 | 9.8 | 2.71 | 3.34 | 6.7 |
| Sum | 1454.6 | 1443.5 | 1654.2 | 1741.7 | 1739.3 | 1667.5 | 1814.7 | 1852.4 | 1837.0 | 2192.1 | 2233.6 | 2275.3 |

| | Examples | | | | | | | | | | |
|---|---|---|---|---|---|---|---|---|---|---|---|
| | | | | | | | | Comparative | | | |
| | Example 9 | | | Example 10 | | | Example 11 | example 1 | Comparative example 2 | | |
| | Test amount | | | | | | | | | | |
| | 2 sprays | | | 2 sprays | | | 2 sprays | 2 sprays | 2 sprays | | |
| | Sampling point | | | | | | | | | | |
| | 0 day | 5 days | 10 days | 0 day | 5 days | 10 days | 0 day | 0 day | 0 day | 5 days | 10 days |
| Adaptor | 1.19 | 0.61 | 1.84 | 4.43 | 2.84 | 6.92 | 14.28 | 1.5 | 5.95 | 5.77 | 6.28 |
| Artificial | 199.38 | 410.14 | 276.38 | 229.03 | 233.36 | 170.67 | 1398.77 | 2790.0 | 569.03 | 623.71 | 455.16 |
| larynx | | | | | | | | | | | |
| stage 1 | 126.58 | 79.86 | 100.44 | 127.72 | 80.52 | 109.5 | 708.72 | 20.6 | 249.03 | 326.95 | 228.79 |
| stage 2 | 498.61 | 441.39 | 505.27 | 557.56 | 374.23 | 533.19 | 1087.1 | 2.1 | 635.51 | 585.76 | 539.06 |
| stage 3 | 489.45 | 562.09 | 580.99 | 535.04 | 603.72 | 587.49 | 525.32 | 0.3 | 447.24 | 380.47 | 447.02 |
| stage 4 | 500.99 | 588.86 | 510.12 | 462.91 | 609.24 | 490 | 329.62 | 0.3 | 335.57 | 316.18 | 345.69 |
| stage 5 | 228.45 | 255.01 | 138.63 | 202.43 | 207.62 | 191.49 | 67.17 | 0.2 | 120.81 | 145.44 | 119.59 |
| stage 6 | 37.01 | 26.84 | 13.36 | 21.4 | 23.64 | 24.43 | 7.64 | 0.1 | 25.42 | 38.09 | 17.37 |
| stage 7 | 3.62 | 2.81 | 1.3 | 2.01 | 0.74 | 2.09 | 0 | 0.1 | 4.52 | 7.71 | 2.15 |
| MOC | 18.1 | 3.32 | 1.9 | 5.34 | 3.04 | 4.68 | 0 | 2.5 | 7.09 | 15.37 | 5.98 |
| Sum | 2103.4 | 2370.9 | 2130.2 | 2147.9 | 2139.0 | 2120.5 | 4138.6 | 2817.7 | 2400.2 | 2445.5 | 2167.1 |

CITDAS analysis software was used, and the statistical analysis of the assay data of each stage of NGI are shown in Table 16.

TABLE 16

| | Spray | Sampling | Total | Fine particle | <5 μm fine particle | MMAD |
|---|---|---|---|---|---|---|
| Examples | times | point | amount (μg) | dose (μg) | proportion (%) | (μm) |
| Example 1 | 2 sprays | 0 days | 307.1 | 162.08 | 52.88 | 4.53 |
| | | 5 days | 244.1 | 144.49 | 59.90 | 3.94 |
| | | 10 days | 191.2 | 128.40 | 67.64 | 3.35 |
| Example 2 | 4 sprays | 0 days | 5003.5 | 862.09 | 17.47 | 7.95 |
| | | 5 days | 5257.8 | 585.60 | 11.33 | 9.15 |
| | | 10 days | 5147.9 | 672.11 | 13.22 | 8.63 |
| Example 3 | 2 sprays | 0 days | 1712.1 | 360.44 | 21.08 | 7.61 |
| | | 5 days | 1715.1 | 335.69 | 19.60 | 8.07 |
| | | 10 days | 1742.9 | 247.12 | 14.19 | 8.75 |
| Example 4 | 2 sprays | 0 days | 1757.5 | 366.94 | 20.91 | 7.54 |
| | | 5 days | 1866.0 | 258.70 | 13.88 | 8.60 |
| | | 10 days | 1713.1 | 287.98 | 16.85 | 7.89 |
| Example 5 | 2 sprays | 0 days | 1454.6 | 651.90 | 44.85 | 5.09 |
| | | 5 days | 1443.5 | 657.36 | 45.56 | 4.74 |
| | | 10 days | 1654.2 | 400.19 | 24.22 | 6.93 |
| Example 6 | 2 sprays | 0 days | 1741.7 | 396.01 | 22.77 | 7.40 |
| | | 5 days | 1739.3 | 261.68 | 15.05 | 8.28 |
| | | 10 days | 1667.5 | 362.33 | 21.76 | 7.46 |
| Example 7 | 2 sprays | 0 days | 1814.7 | 361.64 | 19.97 | 7.84 |
| | | 5 days | 1852.4 | 198.65 | 10.74 | 9.41 |
| | | 10 days | 1837.0 | 268.18 | 14.63 | 8.94 |
| Example 8 | 2 sprays | 0 days | 2192.1 | 847.39 | 38.70 | 5.46 |
| | | 5 days | 2233.6 | 921.91 | 41.31 | 5.20 |
| | | 10 days | 2275.3 | 936.04 | 41.20 | 4.97 |
| Example 9 | 2 sprays | 0 days | 2103.4 | 999.62 | 47.55 | 4.78 |
| | | 5 days | 2370.9 | 1127.07 | 47.55 | 4.44 |
| | | 10 days | 2130.2 | 913.08 | 42.90 | 5.05 |
| Example 10 | 2 sprays | 0 days | 2147.9 | 920.50 | 42.95 | 5.16 |
| | | 5 days | 2139.0 | 1116.72 | 52.28 | 4.43 |
| | | 10 days | 2120.5 | 963.53 | 45.59 | 5.03 |
| Example 11 | 2 sprays | 0 days | 4138.6 | 597.89 | 14.50 | 8.27 |
| Comparative | 2 sprays | 0 days | 2817.7 | 3.32 | 0.12 | NA |
| example 1 | | | | | | |
| Comparative | 2 sprays | 0 days | 2400.2 | 672.56 | 28.09 | 6.38 |
| example 2 | | 5 days | 2445.5 | 676.68 | 27.74 | 6.59 |
| | | 10 days | 2167.1 | 671.79 | 31.09 | 6.02 |

The NGI APSD assay results show that the proportion of fine particles with spray droplet particle size less than 5 μm in the inhalation spray of Semaglutide prepared in Examples 1 to 11 is not less than 10%, and the mass median aerodynamic particle size (MMAD) is not more than 10 μm, which meets the particle size requirements of inhalation administration.

The assay data of Examples 1, 2 and 11 and Comparative example 1 show that the concentration of the medicinal liquid increased from 2% to 10%, 15% and 20%, the proportion of fine particles with spray droplet particle size <5 μm decreased sharply, and the deposition efficiency in the lung is so low that the medicinal liquid at a concentration of 20% is no longer suitable for inhalation administration. Therefore, it is most suitable for inhalation administration to control the concentration of the medicinal liquid below 15%.

Surprisingly, it was found that the spray MMAD of the 7.5% medicinal liquid is 5.09 μm in Example 5, which is equivalent to the spray MMAD of 4.53 μm of 2% medicinal liquid in Example 1, and the proportions of fine particles with particle size <5 μm are 44.85% and 52.88%, respectively, indicating that the addition of absorption enhancer Tween 80 and bacteriostatic benzyl alcohol can effectively reduce the spray particle size of high concentration medicinal liquid. In addition, a higher proportion of fine particles can ensure a higher drug deposition in the lung.

The comparison between Example 2 and Comparative Example 2 showed that the drug concentrations of both are 10%, but when benzyl alcohol is used as bacteriostatic agent, the spray MMAD of the medicinal liquid is 6.38 μm, which is lower than the MMAD of 7.95 μm when benzalkonium chloride is used as the bacteriostatic agent, indicating that benzyl alcohol as bacteriostatic agent can also reduce the spray particle size. The comparison between Examples 8 to 10 and Comparative Example 2 showed that, when 0.02%, 0.10% and 0.15% of Tween 80 were added to the medicinal liquid with benzyl alcohol as bacteriostatic agent, the spray MMADs of the medicinal liquids were reduced to 5.46 μm, 4.78 μm and 5.16 μm, respectively, indicating that the combined use of benzyl alcohol and Tween 80 can further reduce the spray particle size of the medicinal liquid of Semaglutide.

### Study on Pharmacokinetics of Airway Administration in Rats

### Experimental Method

42 SD rats were weighed the day before administration and randomly divided into 7 groups with 6 rats in each group, in which 6 groups were experimental groups, and were given the inhalation spray of Semaglutide of Examples 3 to 7 and Example 12 respectively (in animal experiments, in order to compare more accurately, the main drug concentrations of the sprays are consistent, and especially Example 12 was added: Example 12 is different from Example 1 only in the concentration of the main drug, and other adjuvants, contents and preparation methods are the same as Example 1. The concentration of the main drug in Example 12 was 7.5%, and that in Example 1 was 2%). Group 1 was the control group, which was given Semaglutide injection. The rats were fed continuously and drank freely before the experiment. In each experimental group, the medicinal liquid of inhalation spray of Semaglutide was atomized through an atomization needle and then inhaled via trachea for administration. Firstly, the animals were anesthetized by inhaling isoflurane for a short time, and fixed. The mouth of animal was opened. The oropharynx was pressed with an instrument to expose the trachea, and the pre-filtered medicinal liquid was atomized and sprayed into the trachea through the atomization needle. The control group was administered by subcutaneous injection of Semaglutide injection. 0.2 ml of whole blood was taken from the jugular vein of SD rats before (0 h) and at 0.5, 2, 4, 6, 8, 12, 24, 48 and 72 h after administration, respectively, and the concentration of Semaglutide in rat plasma was quantitatively detected after centrifugation. The specific administration schemes are shown in Table 17:

TABLE 17

| | Experimental groups | | | | | | | |
|---|---|---|---|---|---|---|---|---|
| | Example 12 | | Example 3 | | Example 4 | | Example 5 | |
| Specification | 75 | mg/ml | 75 | mg/ml | 75 | mg/ml | 75 | mg/ml |
| Administration | 30 | μl | 30 | μl | 30 | μl | 30 | μl |
| volume | | | | | | | | |
| Administration dose | 11.25 | mg/kg | 11.25 | mg/kg | 11.25 | mg/kg | 11.25 | mg/kg |

| | | | | |
|---|---|---|---|---|
| Bacteriostatic | phenol | phenol | phenol | benzyl alcohol |
| agent | (5.5 mg/ml) | (5.5 mg/ml) | (5.5 mg/ml) | (10 mg/ml) |
| Absorption | / | sodium octanoate | sucrose laurate | Tween 80 |
| enhancer | | (100 mg/ml) | (10 mg/ml) | (0.2 mg/ml) |

| | |
|---|---|
| Buffer salt, etc. | disodium phosphate, sodium hydroxide and purified water |

| | Experimental groups | | | | | |
|---|---|---|---|---|---|---|
| | Example 6 | | Example 7 | | Control group | |
| Specification | 75 | mg/ml | 75 | mg/ml | 3.0 ml:4 mg | |
| Administration | 30 | μl | 30 | μl | 100 | μl |
| volume | | | | | | |
| Administration | 11.25 | mg/kg | 11.25 | mg/kg | 0.2 | mg/kg |

| | | | |
|---|---|---|---|
| dose | | | |
| Bacteriostatic | benzyl alcohol (10 mg/ml) | benzyl alcohol (10 mg/ml) | phenol |
| agent | | | |
| Absorption | sodium octanoate (100 mg/ml), | sucrose laurate (100 mg/ml), | / |
| enhancer | Tween 80 (0.2 mg/ml) | Tween 80 (0.2 mg/ml) | |

| | |
|---|---|
| Buffer salt, etc. | disodium phosphate, sodium hydroxide and purified water |

### Results

Using WinNonLin 8.3 software, the pharmacokinetic parameters of each group were calculated based on data of plasma concentration according to the non-compartment model method, and the average value of each parameter for 6 animals was calculated. The results are shown in Table 18:

TABLE 18

| | | | | | Relative |
|---|---|---|---|---|---|
| | Tmax | Cmax | AUC | | bioavailability |
| Formulation | (h) | (ng/mL) | (h*ng/mL) | AUC/Dose | (%) |
| Example 12 | 0.09 | 11825 | 227463.36 | 20218.965 | 14.58% |
| Example 3 | 3.4 | 35938.3 | 709500 | 63066.7 | 48.14% |
| Example 4 | 2.3 | 31776.7 | 546750 | 48600 | 37.10% |
| Example 5 | 4.3 | 35941.7 | 768000 | 68266.7 | 52.11% |
| Example 6 | 4.0 | 34516.7 | 661500 | 58800 | 44.89% |
| Example 7 | 3.7 | 25666.7 | 643200 | 47644.4 | 36.37% |
| Control | 11.3 | 984.2 | 26200 | 131000 | / |
| group | | | | | |

The results of pharmacokinetic assays showed that compared with Example 12, the relative bioavailability was significantly improved after the absorption enhancer was added to the medicinal liquid formulation, especially in the Example 5 experimental group, which reached a maximum of 52.11%, showing a better potential for preparation development. By comparing each formulation, it was found that Tween 80 and sodium octanoate showed the most obvious improvement in bioavailability. The peak time of each experimental group was faster than that of the control group, indicating that the absorption of inhaled Semaglutide was faster than that of subcutaneous injection.

1. An inhalation spray of Semaglutide, wherein by weight to volume, Semaglutide accounts for 0.01% to 20% of the inhalation spray, and a proportion of fine particles with a particle size less than 5 μm formed after the inhalation spray is atomized by a spray device is not less than 10%.

2. The inhalation spray according to claim 1, wherein a mass median aerodynamic diameter (MMAD) of the fine particles formed after the inhalation spray is atomized by a spray device is not more than 10 μm.

3. The inhalation spray according to claim 1, wherein by weight to volume, Semaglutide accounts for 0.01% to 15%.

4. The inhalation spray according to claim 1, wherein the inhalation spray further comprises 0.01% to 2% of a pharmaceutically acceptable bacteriostatic agent, by weight to volume,
and the bacteriostatic agent is selected from the group consisting of: phenol, benzyl alcohol, benzalkonium chloride, m-phenol, sodium benzoate, chlorobutanol, butyl hydroxybenzoate, methyl hydroxybenzoate and a combination thereof.

5. The inhalation spray according to claim 1, wherein the inhalation spray further comprises a pharmaceutically acceptable bacteriostatic agent of phenol or benzyl alcohol, which accounts for, by weight to volume,
0.45% to 0.65% of the inhalation spray, when the bacteriostatic agent is phenol; or
0.5% to 1.5% of the inhalation spray, when the bacteriostatic agent is benzyl alcohol.

6. The inhalation spray according to claim 1, wherein the inhalation spray further comprises 0% to 15% of an absorption enhancer, by weight to volume, and
the absorption enhancer is selected from the group consisting of: Tween 20, Tween 80, sodium octanoate, sodium deoxycholate, sodium dodecyl sulfate, sucrose laurate, sodium 8-(2-hydroxybenzamido)octanoate and a combination thereof.

7. The inhalation spray according to claim 1, wherein the inhalation spray further comprises 0.01% to 0.15% of an absorption enhancer of Tween 80, by weight to volume.

8. The inhalation spray according to claim 1, wherein the inhalation spray further comprises a pH buffer selected from the group consisting of disodium phosphate, sodium biphosphate, sodium citrate, sodium acetate, sodium carbonate, sodium tartrate and a combination thereof.

9. The inhalation spray according to claim 1, wherein the inhalation spray has a pH of 6.5 to 8.5.

10. The inhalation spray according to claim 1, wherein the proportion of fine particles with the particle size less than 5 μm formed after the inhalation spray is atomized by the spray device is not less than 20%.

11. The inhalation spray according to claim 1, wherein the proportion of fine particles with the particle size less than 5 μm formed after the inhalation spray is atomized by the spray device is not less than 30%.

12. The inhalation spray according to claim 4, wherein the bacteriostatic agent is selected from the group consisting of phenol, benzyl alcohol, benzalkonium chloride, and a combination thereof.

13. The inhalation spray according to claim 6, wherein the inhalation spray further comprises 0.01% to 10% of an absorption enhancer, by weight to volume, and
the absorption enhancer is selected from the group consisting of Tween 80, sodium octanoate, sucrose laurate and a combination thereof.

14. The inhalation spray according to claim 8, wherein the pH buffer is selected from the group consisting of disodium phosphate, sodium biphosphate, and a mixture thereof.

15. The inhalation spray according to claim 9, wherein the inhalation spray has a pH of 7.1 to 7.7.

16. The inhalation spray according to claim 9, wherein the inhalation spray has a pH of 7.3 to 7.5.

17. A preparation method of the inhalation spray according to claim 1, comprising the steps of:
(1) taking 80% of a formulation volume of water, adding a bacteriostatic agent, an absorption enhancer and a pH buffer, stirring to dissolve, and adjusting pH to 7.1 to 7.7 with sodium hydroxide and hydrochloric acid;
(2) adding Semaglutide and stirring until it is completely dissolved;
(3) adjusting pH to 7.3 to 7.5 with sodium hydroxide and hydrochloric acid; and
(4) adding water to the formulation volume.
